(19) United States
(12) Patent Application Publication
(10) Pub. No.: US 2019/0300460 A1
(43) Pub. Date: Oct. 3, 2019

(54) PROCESS FOR IMPROVING THE PRODUCTION OF A CHLORINATED ALKENE BY CAUSTIC DEYDROCHLORINATION OF A CHLORINATED ALKANE BY RECYCLING

(71) Applicant: BLUE CUBE IP LLC, Clayton, MO (US)

(72) Inventors: Max Tirtowidjojo, Clayton, MO (US); Marc Sell, Clayton, MO (US); John D. Myers, Clayton, MO (US)

(21) Appl. No.: 16/373,248

(22) Filed: Apr. 2, 2019

Related U.S. Application Data
(60) Provisional application No. 62/652,059, filed on Apr. 3, 2018.

(51) Int. Cl. C07C 17/23

(52) U.S. Cl. CPC C07C 17/23

(57) ABSTRACT

The present invention provides processes for the preparation of a chlorinated alkene from a chlorinated alkane using a phase transfer catalyst and an aqueous base.

### CROSS REFERENCE TO RELATED APPLICATIONS

The instant application claims the benefit of U.S. Provisional Patent Application Ser. No. 62/652,059, filed Apr. 3, 2018, which is incorporated herein by reference in its entirety.

### FIELD OF THE INVENTION

The present disclosure generally relates to the preparation of chlorinated alkenes.

### BACKGROUND OF THE INVENTION

Chlorinated alkenes are useful intermediates for many products including agricultural products, pharmaceuticals, cleaning solvents, gums, silicones, and refrigerants. One method for preparing chloroalkenes involves using a catalyst to dehydrochlorinate a chlorinated alkane. Common dehydrochlorination catalysts include Lewis acids, such as FeCl₃ or AlCl₃, which are not complexed with a ligand. The ligand can reduce the reaction rate and yield of the dehydrochlorination reaction. These catalysts have been shown to be useful providing the chlorinated alkenes in good yields. Yet, additional purification protocols are necessary to remove the catalyst from the chlorinated alkene which can inhibit subsequent processes.

Another method for producing a chlorinated alkene comprises contacting the chlorinated alkane with an aqueous base in a dehydrochlorination process. Generally, these processes are efficient, yet they require a co-solvent such as an alcohol to provide miscibility of the organic and aqueous phases. Processes that do not utilize a co-solvent have been developed, but they are inefficient and require additional separation steps, which reduces the yield of the chlorinated alkene.

An improvement to the base dehydrochlorination process described above utilizes a small amount of a phase transfer catalyst. The phase transfer catalyst enables transfer of active species between the organic and aqueous phases and enhances the kinetics of the process. Due to the cost of the phase transfer catalyst, the overall cost for producing a chlorinated alkene on a production scale by adding a phase transfer catalyst is increased. At the completion of the process, the phase transfer catalyst is normally purged to waste.

It would be desirable to develop a process for preparing a chlorinated alkene with increased reaction kinetics, low unit manufacturing cost, high purity, and enables efficient recycle strategies, and in particular, recycling of the phase transfer catalyst.

### SUMMARY OF THE INVENTION

In one aspect, disclosed herein are processes for preparing chlorinated alkenes. In general, the process comprises reacting a mixture of at least one chlorinated alkane, a phase transfer catalyst, and an aqueous base under conditions detailed below. Once the desired chlorinated alkene(s) is prepared, the reactor contents are transferred into at least one separator where at least a part of the organic phase is separated from the aqueous phase. If desired, the organic phase may be dried. The organic phase is distilled into at least two product effluent streams. The first product effluent stream comprises the chlorinated alkene(s) and optionally unreacted chlorinated alkane. The second product effluent stream comprises, the heavy by-products, the phase transfer catalyst, and optionally, the unreacted at least one chlorinated alkane. At least a portion of the second product effluent stream is recycled to the process, thus providing additional efficiencies by reducing the raw material and catalyst used per pound of product produced (unit ratios) and hence reduces the overall manufacturing cost of the process. Additional separations may be employed at any point within the separation system to remove light by-products and/or water from any of the aforementioned streams.

In another aspect, disclosed herein are processes for the preparation of 1,1,3-trichloropropene, 3,3,3-trichloropropene, or combinations thereof. Generally, the process comprises reacting a mixture of 1,1,1,3-tetrachloropropane (250FB), aqueous base, and a phase transfer catalyst under conditions detailed below. Once the desired 1,1,3-trichloropropene, 3,3,3-trichloropropene, or combinations thereof are prepared, the reactor contents are transferred into at least one separator where at least a part of the organic phase comprising 1,1,3-trichloropropene, 3,3,3-trichloropropene, or combinations, unreacted 1,1,1,3-tetrachloropropane, heavy by-products, and the phase transfer catalyst is separated from the aqueous phase. If desired, the organic phase may be further dried. Dissolved water in the organic phase may be removed by conventional distillation or by adsorbing them in a drying bed consisting of alumina or silica or other type of absorbent, or by a combination of both. The organic phase is distilled and at least two product effluent streams are formed. The first product effluent stream comprises 1,1,3-trichloropropene, 3,3,3-trichloropropene, or combinations thereof and optionally 1,1,1,3-tetrachloropropane. If desired, this effluent stream may be further distilled or purified, to obtain material of the desired purity. For example, this effluent stream may be distilled to produce two additional product effluent streams, and overhead effluent stream comprising mixtures of 1,1,3-trichloropropene and 3,3,3-trichloropropene and a bottom effluent stream comprising heavy byproducts, the phase transfer catalyst, and optionally, 1,1,1,3-tetrachloropropane. If desired, at least a portion of the bottom effluent stream may be recycled to the reaction mixture. The second product stream comprises the heavy by-products, the phase transfer catalyst, and optionally, unreacted 1,1,1,3-tetrachloropropane. The second product effluent stream may be further distilled to produce an overhead stream comprising 1,1,1,3-tetrachloropropane and a bottom stream comprising heavy by-products and phase transfer catalyst. The second product effluent stream, the overhead stream, a portion of the bottom stream, or combinations thereof are recycled into the process to enhance the kinetics, and reduce the raw material and catalyst unit ratios and hence the overall manufacturing cost of the process.

In another aspect, disclosed herein are processes for the preparation of 1,1,2,3-tetrachloropropene, 2,3,3,3-tetrachloropropene, or combinations thereof. Generally, the process comprises reacting a mixture of 1,1,1,2,3-pentachloropropane (240DB), aqueous base, and a phase transfer catalyst under conditions detailed below. Once the desired 1,1,2,3-tetrachloropropene, 2,3,3,3-tetrachloropropene, or combinations thereof is prepared, the reactor contents are transferred into at least one separator where at least a part of the organic phase comprising 1,1,2,3-tetrachloropropene, 2,3,3,3-tetrachloropropene, or combinations thereof, unreacted 1,1,1,2,3-pentachloropropane, heavy by-products, and the phase transfer catalyst is separated from the aqueous phase. If desired, the organic phase may be further dried. Dissolved water in the organic phase may be removed by conventional distillation or by adsorbing them in a drying bed consisting of alumina or silica or other type of absorbent, or by a combination of both. The organic phase is distilled and at least two product effluent streams are formed. The first product effluent stream comprises 1,1,2,3-tetrachloropropene, 2,3,3,3-tetrachloropropene, or combinations thereof, and optionally 1,1,1,2,3-pentachloropropane. If desired, this first product effluent stream may be further distilled to produce two additional product effluent streams, an overhead effluent stream comprising 1,1,2,3-tetrachloropropene, 2,3,3,3-tetrachloropropene, or combinations thereof and a bottom effluent stream, which may contain 1,1,1,2,3-pentachloropropane. Still further, at least a portion of the bottom effluent stream may be recycled to the reaction mixture. The second product effluent stream comprises the heavy by-products, the phase transfer catalyst, and optionally, unreacted 1,1,1,2,3-pentachloropropane (240 DB). If desired, at least a portion of this second product effluent stream may be recycled to the reaction mixture, wherein the reaction temperature is about 40° C. to about 120° C. The second product effluent stream may be further distilled to produce an overhead stream, which may contain 1,1,1,2,3-pentachloropropane and a bottom stream comprising heavy by-products and phase transfer catalyst. If desired, the overhead stream and/or at least a portion of the bottom stream may be recycled to the reaction mixture. In general, the second product effluent stream, the overhead stream, or a portion of the bottom stream, or combinations thereof, may be recycled into the process to enhance the kinetics, and reduce the raw material and catalyst unit ratios and the overall manufacturing cost of the process.

In another aspect, disclosed herein are processes for the preparation of 1,1,3,3-tetrachloropropene, 1,3,3,3-tetrachloropropene, or combinations thereof. Generally, the process comprises reacting a mixture of 1,1,1,3,3-pentachloropropane (240FA), an aqueous base, and a phase transfer catalyst under conditions detailed below. Once the desired 1,1,3,3-tetrachloropropene, 1,3,3,3-tetrachloropropene, or combinations thereof is prepared, the reactor contents are transferred into at least one separator where at least a part of the organic phase comprising 1,1,3,3-tetrachloropropene, 1,3,3,3-tetrachloropropene, or combinations thereof, unreacted 1,1,1,3,3-pentachloropropane, heavy by-products, and the phase transfer catalyst is separated from the aqueous phase. If desired, the organic phase may be further dried. Dissolved water in the organic phase may be removed by conventional distillation or by adsorbing them in a drying bed consisting of alumina or silica or other type of absorbent, or by a combination of both. The organic phase is distilled and at least two product effluent streams are formed. The first product effluent stream comprises 1,1,3,3-tetrachloropropene, 1,3,3,3-tetrachloropropene, or combinations thereof and optionally 1,1,1,3,3-pentachloropropane (240FA). If desired, the first product effluent stream may be further distilled to produce two additional product effluent streams, an overhead effluent stream comprising 1,1,3,3-tetrachloropropene, 1,3,3,3-tetrachloropropene, or combinations thereof and a bottom effluent stream which may contain 1,1,1,3,3-pentachloropropane. At least a portion of the bottom effluent stream may be recycled to the reaction mixture. The second product effluent stream comprises heavy by-products, the phase transfer catalyst and optionally 1,1,1,3,3-pentachloropropane (240FA). This second product stream may be further distilled to produce an overhead stream which may contain unreacted 1,1,1,3,3-pentachloropropane and a bottom stream comprising the heavy by-products and the phase transfer catalyst. At least a portion of the bottom stream may be recycled to the reactor. Further, the reaction temperature may be about 40° C. to about 120° C. or 80° C. to 100° C. The second product stream, the overhead stream, a portion of the bottom stream, or combinations thereof are recycled into the process to enhance the kinetics, and reduce the raw material and catalyst unit ratios and the overall manufacturing cost of the process.

In another aspect, disclosed herein are processes for the preparation of 1,1,2,3-tetrachloropropene. Generally, the process comprises reacting a mixture of 1,1,2,2,3-pentachloropropane, aqueous base, and a phase transfer catalyst under conditions detailed below. Once the desired 1,1,2,3-tetrachloropropene is prepared, the reactor contents are transferred into at least one separator where at least a part of the organic phase comprising 1,1,2,3-tetrachloropropene, unreacted 1,1,2,2,3-pentachloropropane, heavy by-products, and the phase transfer catalyst is separated from the aqueous phase. If desired, the organic phase may be dried. The organic phase is distilled and at least two product effluent streams are formed. The first product effluent stream comprises 1,1,2,3-tetrachloropropene, and may further contain 1,1,2,2,3-pentachloropropane. The second product effluent stream comprises unreacted 1,1,2,2,3-pentachloropropane, the heavy by-products, and phase transfer catalyst. The second product effluent stream may be further distilled to produce an overhead stream comprising 1,1,2,2,3-pentachloropropane and a bottom stream comprising heavy by-products and phase transfer catalyst. The second product stream, the overhead stream, or a portion of the bottom stream, or combinations thereof are recycled into the process to enhance the kinetics, and reduce the raw material and catalyst unit ratios and hence the overall manufacturing cost of the process. Other features and iterations of the invention are described in more detail below.

### BRIEF DESCRIPTION OF FIGURES

The following figures illustrate non-limiting embodiments of the present invention:

FIG. 1 is a graphical representation showing the % selectivity to the desired 1,1,3-trichloropropene, 3,3,3-trichloropropene, or combinations thereof versus the % conversion of the 1,1,1,3-tetrachloropropane using either fresh Aliquat 336 or recycled heavy by-products and phase transfer catalyst at various mixing speeds.

FIG. 2 is a graphical representation of the apparatus used in the process for the production of 1,1,3-trichloropropene, 3,3,3-trichloropropene, or combinations thereof from 1,1,1,3-tetrachloropropane, an aqueous solution of NaOH with NaCl, and Aliquat 336.

FIG. 3 is graphical representation showing the reaction kinetics of the process using fresh Aliquat 336 and recycled heavy by-product and Aliquat 336 at various mixing speeds.

### DETAILED DESCRIPTION OF THE INVENTION

In one aspect, the processes for preparing chlorinated alkenes comprise contacting at least one chlorinated alkane, a phase transfer catalyst, and an aqueous base. The products from the dehydrochlorination are separated and a portion of the product streams comprising heavy by-products and phase transfer catalyst is recycled into the process.

### (I) Processes for Preparing Chlorinated Alkenes

The process for preparing a chlorinated alkene comprises a) preparing and reacting a mixture comprising at least one chlorinated alkane, an aqueous base, and a phase transfer catalyst wherein the mixture comprises an aqueous phase and an organic phase; b) separating the aqueous and organic phases and optionally drying the organic phase; c) distilling the organic phase to not only isolate a product stream comprising the chlorinated alkene from a product stream comprising the chlorinated alkane but also to produce a third product stream comprising heavy by-products and the phase transfer catalyst. Under process conditions described below, a high yield of the chlorinated alkene results.

Compared to other dehydrochlorination processes, recycling a part of a product effluent steam comprising heavy by-products and phase transfer catalyst into step (a) provides similar % conversion as compared to fresh phase transfer catalyst, increased reaction kinetics, and recycling/utilization of a valuable material which normally would be purged to waste. This result is unpredicted and unexpected.

(a) chlorinated Alkane

The at least one chlorinated alkane useful in this process may be a dichloropropane, trichloropropane, a tetrachloropropane, a pentachloropropane, a hexachloropropane, or combinations thereof. Non-limiting examples of trichloropropanes, tetrachloropropanes, pentachloropropanes, and hexachloropropanes include, but are not limited to 1,1-dichloropropane; 1,2-dichloropropane; 1,3-dichloropropane; 1,1,1-trichloropropane; 1,1,2-trichloropropane; 1,2,2-trichloropropane; 1,2,3-trichloropropane; 1,1,1,2-tetrachloropropane; 1,1,2,2-tetrachloropropane; 1,1,1,3-tetrachloropropane; 1,1,2,3-tetrachloropropane; 1,1,3,3-tetrachloropropane; 1,1,1,2,3-pentachloropropane; 1,1,1,2,2-pentachloropropane, 1,1,2,3,3-pentachloropropane; 1,1,2,2,3-pentachloropropane; 1,1,1,3,3-pentachloropropane; 1,1,1,3,3,3-hexachloropropane; 1,1,2,2,3,3-hexachloropropane; or combinations thereof.

One method for preparing these chlorinated alkanes is through the telomerization process. In this process, carbon tetrachloride (Tet), an alkene or chlorinated alkene, a catalyst system comprising metallic iron, ferric chloride, and/or ferrous chloride, and a trialkylphosphate and/or a trialkylphosphite are contacted to produce the chlorinated alkanes. As an illustrative example, using ethylene as the monomer in the above described telomerization process yields tetrachloropropanes or pentachloropropanes. Utilizing vinyl chloride as the monomer, pentachloropropanes would result. The skilled artisan readily knows other methods for preparing chlorinated alkanes. In various embodiment, the at least one chlorinated alkane comprises a tetrachloropropane. In a preferred embodiment, the at least one chlorinated alkane comprises 1,1,1,3-tetrachloropropane, also known as 250FB. In another preferred embodiment, the at least one chlorinated alkane comprises 1,1,1,2,3-pentachloropropane, also known as 240DB. In still another preferred embodiment, the at least one chlorinated alkane comprises 1,1,1,3,3-pentachloropropane, also known as 240FA.

The chlorinated alkane may be crude, unpurified product from the telomerization reaction, partially purified, or fully purified by means known to the skilled artisan. One common method of purification the chlorinated alkane is distillation. Non-limiting examples of distillations may be a simple distillation, flash distillation, a fractional distillation, a steam distillation, or a vacuum distillation.

Generally, the chlorinated alkane useful in the process may have a purity greater than 10 wt %. In various embodiments, the purity of the chlorinated alkane may have a purity greater than 10 wt %, greater than 30 wt %, greater than 50 wt %, greater than 75 wt %, greater than 90 wt %, greater than 95 wt %, or greater than 99 wt %.

(b) phase Transfer Catalyst

A wide variety of phase transfer catalyst may be used in the dehydrochlorination process of the at least one chlorinated alkane to produce chlorinated alkenes. The phase transfer catalyst is soluble in the chlorinated alkane, the chlorinated alkene, or combinations thereof. Non-limiting examples of phase transfer catalysts may be quaternary ammonium salts, phosphonium salts, and pyridinium salts. In some embodiments, the phase transfer catalyst may be a quaternary ammonium salt. Non-limiting examples of suitable salts are chlorides, bromides, iodides, or acetates. Non-limiting examples of quaternary ammonium salts include trioctylmethylammonium chloride (Aliquat 336), trioctylmethylammonium bromide, dioctyldimethylammonium chloride, dioctyldimethylammonium bromide, Arquad 2HT-75, benzyldimethyldecylammonium chloride, benzyldimethyldecylammonium bromide, benzyldimethyldecylammonium iodide, benzyldimethyltetradecylammonium chloride, dimethyldioctadecylammonium chloride, dodecyltrimethylammonium chloride, tetrabutylammonium chloride, tetrabutylammonium bromide, tetrabutylammonium iodide, tetrabutylammonium acetate, tetrahexylammonium chloride, tetraoctylammonium chloride, tridodecylmethylammonium chloride, tetraethylammonium chloride, tetraethylammonium bromide, tetraethylammonium iodide, or combinations thereof. In some embodiments, more than one phase transfer catalyst is used. In a preferred embodiment, the phase transfer catalyst is trioctylmethylammonium chloride (Aliquat 336).

Generally, the amount of the phase transfer catalyst may range from 0.001 wt % to about 10.0 wt % based on the total weight of the components. In various embodiments, the amount of the phase transfer catalyst may range from 0.001 wt % to about 10.0 wt %, from 0.05 wt % to 7.5 wt %, from 0.02 wt % to about 2.5 wt %, or from 0.01 wt % to about 1.0 wt %.

(c) Aqueous Base

The dehydrochlorination process utilizes an aqueous base. In an embodiment, the aqueous base may be an inorganic base. The aqueous base may further contain an inorganic halide salt. In an embodiment, the aqueous phase comprising an aqueous base may be produced by the chloroalkali process.

The inorganic base may be an alkali or alkali earth metal base. Non-limiting examples of these alkali or alkali earth bases may be LiOH, NaOH, KOH, Ba(OH)₂, Ca(OH)₂, Na₂CO₃, K₂CO₃, NaHCO₃, KHCO₃, or combinations thereof . In a preferred embodiment, the alkali or alkali earth metal base may be NaOH, KOH, or combinations thereof. Still more preferably, the base comprises NaOH.

The halide salt may be any alkali or alkali earth metal halide salt. Non-limiting examples of these alkali or alkali earth metal salt halide salts may be selected from a group consisting of lithium chloride, sodium chloride, potassium chloride, barium chloride, calcium chloride, or combinations thereof. In a preferred embodiment, the chloride salt is sodium chloride. In another embodiment, an aqueous base comprises a mixture of NaOH and at least one chloride salt which was produced from the chloroalkali process through the electrolysis of sodium chloride in a diaphragm cell. In other embodiments, the concentration of the alkali or alkali earth metal halide salt is up to or greater than the saturation limit of the alkali or alkali earth metal halide salt in the inorganic base.

Generally, the concentration of the aqueous base may range from 5 wt % to about 50 wt %. In various embodiments, the concentration of the aqueous base may range from 5 wt % to about 50 wt %, from 7 wt % to about 40 wt %, from 9 wt % to about 30 wt %, or from 10 wt % to about 20 wt %. In a preferred embodiment, the concentration of the aqueous base may range from 5 wt % to about 10 wt %.

In general, the mole ratio of the base(s) to the chlorinated alkane may range from 0.1:1.0 to about 2.0:1.0. In various embodiments, the mole ratio of the base(s) to the chlorinated alkane may range from 0.1:1.0 to about 2.0:1.0, from 0.5:1.0 to about 1.5:1.0, or from 0.9:1.0 to about 1.1:1.0. In a preferred embodiment, the mole ratio of the aqueous base to the chlorinated alkane may be about 1.0:1.0.

In general, the concentration of the halide salt may be up to or below the saturation limit. In various embodiments, the concentration of the halide salt may be greater than 0.01 wt %, greater than 1 wt %, greater than 10 wt %, greater than 20 wt %, at the saturation limit for the suitable halide salt, or below the saturation limit for the suitable halide salt.

(d) Reaction Conditions

In general, the dehydrochlorination process for producing a chlorinated alkene includes carrying out the dehydrochlorination reaction in liquid phase at process conditions to enable the preparation of an effective high yield of the chlorinated alkene product.

The process commences by contacting the at least one chlorinated alkane (either purified, partially purified, or unpurified), an aqueous base, and a phase transfer catalyst. The components of the process may be added in any order. All the components of the process are typically mixed at a temperature enabling the preparation of effective high yield of the chlorinated alkene product.

The temperature of the process can and will vary depending on purity of the at least one chlorinated alkane, the phase transfer catalyst, the base, and the concentration of the base. Generally, the temperature of the process may be generally from 20° C. to about 120° C. In various embodiments, the temperature of the process may be generally from 20° C. to about 120° C., from 40° C. to about 80° C., or from 50° C. to 70° C.

In general, the pressure of the process may be greater than 0 psig. In various embodiments, the pressure of the process may be greater than 0 psig, greater than 50 psig, greater than 100 psig, greater than 1000 psig, or greater than 200 psig. In a preferred embodiment, the pressure of the process may be about atmospheric pressure and the process may be conducted under an inert atmosphere such as nitrogen, argon, or helium.

Generally, the reaction is allowed to proceed for a sufficient period of time until the reaction is complete, as determined by any method known to the skilled artisan, such as chromatography (e.g., GC). The duration of the reaction may range from about 5 minutes to about 12 hours. In some embodiments, the duration of the reaction may range from about 5 minutes to about 10 hours, from about 30 minutes to about 9 hours, from about 1 hour to about 8 hours, or from about 4 hours to about 7 hours.

As appreciated by the skilled artisan, the above process may be run in a batch mode or a continuous mode. In another embodiment, the process in continuous modes may be stirred in various methods to improve the mixing of the biphasic system as appreciated by the skilled artisan. One preferred method for ensuring the biphasic contents of the reactor are adequately mixed may be utilizing a jet stirred reactor which mixes the contents of the reactor without an impeller, i.e., jet mixing. The jet mixing is caused by feeding fresh liquid feed, product effluent stream, a recycle stream or combinations thereof to at least one nozzle. In this jet stirred reactor system, the liquid materials comprising of internal recycle and fresh feed are transported vertically or tangentially through the reactor by means of an external pump. A portion of the reaction product is recycled back to the reactor while the rest is removed from the reaction system into the purification step.

The chlorinated alkane fed to the above described process may be converted to the chlorinated alkene isomers in at least 50% conversion. In various embodiments, the conversion of chlorinated alkane to the chlorinated alkene isomers may be at least 50%, at least 60%, at least 75%, at least 85%, at least 95%, and at least 99%. In one embodiment, the chlorinated alkane is converted into the chlorinated alkene at less than 98%.

The selectivity to the desired chlorinated alkene can and will vary depending on the reaction conditions, base, and the purity level of the chlorinated alkane utilized. Generally, the selectivity to the chlorinated alkene may be greater than 70%. In various embodiments, the selectivity to the desired chlorinated alkene may be greater than 70%, greater than 80%, greater than 90%, or greater than 95%. In preferred embodiments, the selectivity to the desired chlorinated alkenes may range from 95% to 99%.

### (II) Separating Chlorinated Alkene Products and Recycling Product Streams

The next step in the process comprises separating purified chlorinated alkenes from the reaction mixture comprising the chlorinated alkene, salt, water, lighter by products, heavier by products, phase transfer catalyst, and unreacted chlorinated alkane starting material. Depending on the purity of the chlorinated alkane used in the process, further components in the reaction mixture may be a trialkylphosphate, a trialkylphosphite, and iron hydroxide. In a preferred embodiment, the chlorinated alkene product comprises a mixture of 1,1,3-trichloropropene, 3,3,3-trichloropropene, or combinations thereof. In another preferred embodiment, the chlorinated alkene product comprises 1,1,2,3-tetrachloropropene, 2,3,3,3-tetrachloropropene, or combinations thereof. In still another preferred embodiment, the chlorinated alkene product comprises 1,1,3,3-tetrachloropropene, 1,3,3,3-tetrachloropropene, or combinations thereof.

The separation process commences by transferring the reaction mixture into a separator or multiple separators. As appreciated by the skilled artisan, many separation techniques may be useful. Non-limiting examples of separation techniques may be decantation, settling, filtration, separation, centrifugation, thin film evaporation, simple distillation, vacuum distillation, fractional distillation, or a combination thereof. The distillations may comprise at least one theoretical plate. Depending on the quality and purity of the chlorinated alkane, various separation processes may be employed in various orders.

The reaction mixture is transferred to the separation device resulting in an aqueous phase comprising the salt and optionally iron hydroxide and an organic phase comprising the chlorinated alkene, unreacted chlorinated alkane, lighter by-products, heavier by-products, phase transfer catalyst, and optionally trialkylphosphate or trialkylphosphite. At least some of the organic phase from the reaction mixture is separated from the aqueous phase from the reaction mixture. In the separation vessel, the aqueous phase can be withdrawn from near or at the top and the organic phase can be withdrawn from near the bottom of said vessel.

The organic phase is optionally further dried and then transferred into a second separator. In an embodiment, the second separator may utilize at least one simple distillation, at least one vacuum distillation, at least one fractional distillation, or combinations thereof. The distillations may comprise at least one theoretical plate. In an embodiment, further drying of any of the organic phases described herein may be accomplished by distilling the organic phase, adding it to an adsorption bed, treating it with molecular sieves or a combination of two or more thereof. In another embodiment, further drying of any of the organic phases described herein may be accomplished by distilling the organic phase, adding it to an adsorption bed, or a combination thereof.

The optional drying may be performed using methods known in the art. Examples include conventional distillation or by adsorbing them in a drying bed consisting of alumina or silica or other type of absorbent, or by a combination of both. Other absorbents include chemical reagents such as Na₂SO₄, P₂O₅, and combinations thereof. Molecular sieves may also be used.

As appreciated by the skilled artisan, separating the purified chlorinated alkene from the organic phase produces at least two product effluent streams wherein one product effluent stream comprises the lighter by-products and the chlorinated alkene, while the other effluent product stream comprises unreacted chlorinated alkane, heavier by-products, phase transfer catalyst, and optionally trialkylphosphate or trialkylphosphite. In various embodiments, separating the purified chlorinated alkene may produce three, four, or more product streams depending on the separation device utilized. To be clear, additional separations may be employed at any point within the separation system to remove light by-products and/or water from any of the streams generated using the processes disclosed herein. As an example, the separation of the chlorinated alkene from the contents of the reactor using two product effluent streams is shown below.

The organic phase may be distilled to produce two product effluent streams, product effluent streams (i) and (ii). Product effluent stream (i) comprises the chlorinated alkene and optionally unreacted chlorinated alkane, while product effluent stream (ii) comprises the heavy by-products, the phase transfer catalyst and optionally, unreacted chlorinated alkane.

Generally, product effluent streams (i) comprising the chlorinated alkene and optionally unreacted chlorinated alkane may be further purified producing two additional product effluent streams (iii) and (iv) wherein product effluent stream (iii) obtained as an overhead stream comprises the chlorinated alkene and product effluent stream (iv), obtained as the bottom stream, comprising the unreacted chlorinated alkane (if present) and heavies. Product effluent stream (ii) may be further purified producing two additional product effluent streams (v) and (vi) wherein product effluent stream (v) comprises the unreacted chlorinated alkane and product effluent stream (vi) comprises heavy by-products and the phase transfer catalyst.

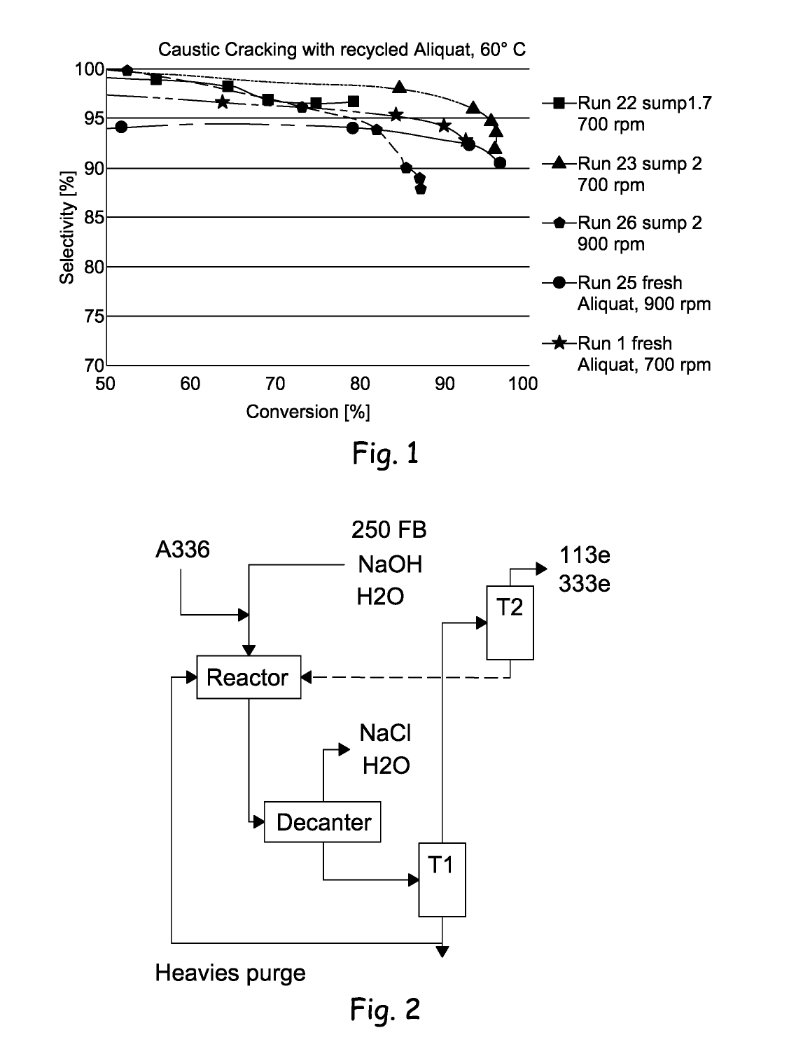

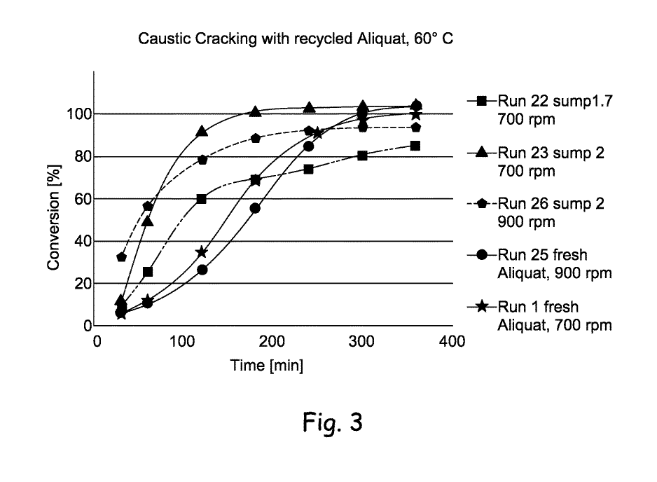

In order to improve the efficiency of the process, various product effluent streams may be externally recycled back into the process. In various embodiments, at least a portion of the product effluent stream (ii) comprising, heavy by-products, the phase transfer catalyst and optionally, unreacted chlorinated alkane, product effluent stream (v) comprising unreacted chlorinated alkane, and product stream (vi) comprising heavy by-products and phase transfer catalyst may be recycled into the dehydrochlorination process, as described above. If desired, the recycle stream, or a portion thereof, is heated using a heat exchanger or other methods known in the art, to maintain the desired temperature, such as the temperature in the reactor. It was unexpectedly found that recycling a portion product effluent stream (ii) and/or product effluent stream (vi) showed comparable % conversion as compared to fresh phase transfer catalyst and an increased reaction rate. FIGS. 1 and 3 show the results of comparable % conversion and enhanced reaction rate.

FIG. 2 shows the process flow diagram of the above recycle product streams. Product effluent streams (ii) and/or (vi) are used to help catalyzed the chlorinated alkane caustic dehydrochlorination. In this process, chlorinated alkane and caustic solution (which may contain NaCl) are mixed into the reactor with a fresh liquid feed comprising phase transfer catalyst. Product effluent stream (ii) from the bottom of T2 which contain mostly unreacted chlorinated alkane, heavy by-products, and phase transfer catalyst recycle from the bottom stream of T1. The product of the reactor is fed to a decanter, where the aqueous phase comprising water and byproduct NaCl is separated as the top layer from the bottom layer organic phase, which comprises the chlorinated alkene, unreacted chlorinated alkane, heavy by-products, and phase transfer catalyst. The organic phase is sent to the first distillation T1 where the product effluent stream (i) comprising unreacted chlorinated alkane and chlorinated alkene product is removed in the overhead stream. T1 reboiler temperature can be 120° C. to 180° C. or higher depending on the pressure used in T1. A portion of the product effluent stream (ii) as the bottom stream may be purged to avoid accumulation of heavy by-products, and the remaining portion of product stream (ii) is recycled to the reactor. The overhead stream of T1 is then fed to column T2 where the chlorinated alkene (product effluent stream (iii)) is purified and recovered in the overhead stream. The bottom stream (product effluent stream (iv)) of T2 containing chlorinated alkane is recycled back to the reactor.

In another embodiment, at least a portion of product effluent stream (ii) and/or (vi) may be mixed with fresh liquid feed (comprising non-recycled chlorinated alkane, phase transfer catalyst, and/or aqueous base) before being recycled back into the reactor in batch mode or continuous mode. In various embodiments, the product effluent streams and fresh liquid feeds may be introduced into the reactor separately or mixed together before entering the process. To be clear, fresh feed streams may contain all or less than all of the following: chlorinated alkane, the phase transfer catalyst, and an aqueous base. The introduction of these fresh liquid feeds into the reactor or mixing the recycle streams with fresh liquid feeds increases the efficiency of the process, reduces the overall cost, maintains the kinetics, maintains the reaction conversion, increase the through-put, and reduces the by-products produced by the process. The amounts of the product effluent streams recycled to the reactor or fresh liquid feeds added to the reactor may be the same or different. One way to measure the amount of product effluent streams and/or fresh liquid feeds being added to the reactor is to identify the mass flow of the materials. The product effluent stream being recycled to the reactor has a product effluent stream mass flow, while the fresh liquid feeds being added to the reactor has a fresh liquid feed mass flow. Mass flows may be measured using methods known in the art.

Generally, the mass of the product effluent stream mass flow being recycled to the fresh liquid feed mass flow is adjusted to not only maintain the conversion of the process but also maintain the kinetics of the process.

Product effluent stream (i) from the first separator comprising the at least one chlorinated alkene produced in the dehydrochlorination process may have a yield of at least about 10%. In various embodiments, product effluent stream (i) comprising at least one chlorinated alkene produced in the process may have a yield of at least about 20%, at least about 50%, at least about 70%, at least about 75%, at least about 80%, at least about 85%, at least about 90%, at least about 95%, or at least about 99%.

While the above describes distilling the organic phase into two product effluent streams, it must be emphasized that the skilled person will readily appreciate the organic phase may be distilled into three or more streams. For example a lights stream, a mid-stream and a heavies stream can readily be prepared by distilling the organic phase or any of the phases disclosed herein. The exact distillation/separation protocols to be used are readily apparent to the skilled practitioner.

### (III) Preferred Embodiment: Process for Preparing of 1,1,3-Trichloropropene, 3,3,3-Trichloropropene, or Combinations Thereof.

(a) Process for Preparing 1,1,3-trichloropropane, 3,3,3-trichloropropane, or Combinations Thereof

In another aspect, disclosed herein are processes for preparing 1,1,3-trichloropropene; 3,3,3-trichloropropene or combinations thereof. The process commences by preparing and reacting a mixture comprising 1,1,1,3-tetrachloropropane; an aqueous base; and a phase transfer catalyst. The phase transfer catalyst is described above in Section (I)(b) and the aqueous base is described above in Section (I)(c). In a preferred embodiment, the phase transfer catalyst is trioctylmethylammonium chloride (Aliquat 336) and the aqueous base comprises 5 to 10 wt % NaOH, KOH, or combinations thereof and up to or below the saturation limit of the corresponding salt.

(b) Reaction Conditions

The reaction conditions are described above in Section (I)(d).

(c) Output From the Process to Prepare 1,1,3-trichloropropene, 3,3,3-trichloropropene, or Combinations Thereof.

The 1,1,1,3-tetrachloropropane fed to the above described process may be converted to 1,1,3-trichloropropene, 3,3,3-trichloropropene, or combinations thereof in at least 50% conversion. In various embodiments, the conversion of 1,1,1,3-tetrachloropropane to 1,1,3-trichloropropene; 3,3,3-trichloropropene or combinations thereof may be at least 50%, at least 60%, at least 75%, at least 85%, at least 95%, and at least 99%.

The selectivity to 1,1,3-trichloropropene; 3,3,3-trichloropropene or combinations thereof can and will vary depending on the reaction conditions, base, the purity level of the 1,1,1,3-tetrachloropropane utilized, and the 1,1,3-trichloropropene; 3,3,3-trichloropropene, or combinations thereof produced. Generally, the selectivity to 1,1,3-trichloropropene; 3,3,3-trichloropropene, or combinations thereof may be greater than 70%. In various embodiments, the selectivity to the 1,1,3-trichloropropene; 3,3,3-trichloropropene, or combinations thereof may be greater than 70%, greater than 80%, greater than 90%, or greater than 95%. In preferred embodiments, the selectivity to the 1,1,3-trichloropropene; 3,3,3-trichloropropene or combinations thereof may range from 95% to 99%.

(d) Separation of the 1,1,3-trichloropropene, 3,3,3-trichloropropene, or Combinations Thereof and Recycling Product Streams.

The process for separating the 1,1,3-trichloropropene; 3,3,3-trichloropropene, or combinations thereof from the reactor contents is described above in Section (II). Specific recycle streams useful in improving the efficiency of the process are described above in Section (II).

The product effluent stream from the separator comprising the 1,1,3-trichloropropene; 3,3,3-trichloropropene, or combinations thereof produced in the dehydrochlorination process may have a yield of at least about 10%. In various embodiments, the product effluent stream comprising 1,1,3-trichloropropene; 3,3,3-trichloropropene, or combinations thereof produced in the process may have a yield of at least about 20%, at least about 50%, at least about 70%, at least about 75%, at least about 80%, at least about 85%, at least about 90%, at least about 95%, or at least about 99%.

### (IV) Preferred Embodiment: Process for Preparing of 1,1,2,3-Tetrachloropropene, 2,3,3,3-Tetrachloropropene, or Combinations Thereof

(a) Process for Preparing 1,1,2,3-tetrachloropropene, 2,3,3,3-tetrachloropropene, or Combinations Thereof.

In yet another aspect, disclosed herein are processes for preparing 1,1,2,3-tetrachloropropene, 2,3,3,3-tetrachloropropene, or combinations thereof. The process commences by preparing and reacting a mixture comprising 1,1,1,2,3-pentachloropropane (240DB); an aqueous base; and a phase transfer catalyst. The phase transfer catalyst is described above in Section (I)(b) and the aqueous base is described above in Section (I)(c). In a preferred embodiment, the phase transfer catalyst is trioctylmethylammonium chloride (Aliquat 336) and the aqueous base comprises 5 to 10 wt % NaOH, KOH, or combinations thereof and up to or below the saturation limit of the corresponding salt.

(b) Reaction Conditions

The reaction conditions are described above in Section (I)(d).

(c) Output From the Process to Prepare 1,1,2,3-tetrachloropropene, 2,3,3,3-tetrachloropropene, or Combinations Thereof.

The 1,1,1,2,3-pentachloropropane fed to the above described process may be converted to 1,1,2,3-tetrachloropropene, 2,3,3,3-tetrachloropropene, or combinations thereof in at least 50% conversion. In various embodiments, the conversion of 1,1,1,2,3-pentachloropropane to 1,1,2,3-tetrachloropropene, 2,3,3,3-tetrachloropropene, or combinations thereof may be at least 50%, at least 60%, at least 75%, at least 85%, at least 95%, and at least 99%.

The selectivity to 1,1,2,3-tetrachloropropene, 2,3,3,3-tetrachloropropene, or combinations thereof can and will vary depending on the reaction conditions, base, the purity level of the 1,1,1,2,3-pentachloropropane, and the 1,1,2,3-tetrachloropropene, 2,3,3,3-tetrachloropropene, or combinations thereof produced. Generally, the selectivity to 1,1,2,3-tetrachloropropene, 2,3,3,3-tetrachloropropene, or combinations thereof may be greater than 70%. In various embodiments, the selectivity to the 1,1,2,3-tetrachloropropene, 2,3,3,3-tetrachloropropene, or combinations thereof may be greater than 70%, greater than 80%, greater than 90%, or greater than 95%. In preferred embodiments, the selectivity to the 1,1,2,3-tetrachloropropene, 2,3,3,3-tetrachloropropene, or combinations thereof may range from 95% to 99%.

(d) Separation of the 1,1,2,3-tetrachloropropene, 2,3,3,3-tetrachloropropene, or Combinations Thereof and Recycling Product Streams.

The process for separating the 1,1,2,3-tetrachloropropene, 2,3,3,3-tetrachloropropene, or combinations thereof from the reactor contents is described above in Section (II). Specific recycle streams useful in improving the efficiency of the process are described above in Section (II).

The product effluent stream from the separator comprising the 1,1,2,3-tetrachloropropene, 2,3,3,3-tetrachloropropene, or combinations thereof produced in the dehydrochlorination process may have a yield of at least about 10%. In various embodiments, the product effluent stream comprising 1,1,2,3-tetrachloropropene, 2,3,3,3-tetrachloropropene, or combinations thereof produced in the process may have a yield of at least about 20%, at least about 50%, at least about 70%, at least about 75%, at least about 80%, at least about 85%, at least about 90%, at least about 95%, or at least about 99%.

### (V) Preferred Embodiment: Process for Preparing of 1,1,3,3-Tetrachloropropene, 1,3,3,3-Tetrachloropropene, or Combinations Thereof.

(a) process for preparing 1,1,3,3-tetrachloropropene, 1,3,3,3-tetrachloropropene, or combinations thereof

In still another aspect, disclosed herein are processes for preparing 1,1,3,3-tetrachloropropene, 1,3,3,3-tetrachloropropene, or combinations thereof. The process commences by preparing and reacting a mixture comprising 1,1,1,3,3-pentachloropropane (240FA); an aqueous base; and a phase transfer catalyst. The phase transfer catalyst is described above in Section (I)(b) and the aqueous base is described above in Section (I)(c). In a preferred embodiment, the phase transfer catalyst is trioctylmethylammonium chloride (Aliquat 336) and the aqueous base comprises 5 to 10 wt % NaOH, KOH, or combinations thereof and up to or below the saturation limit of the corresponding salt.

(b) Reaction Conditions

The reaction conditions are described above in Section (I)(d).

(c) Output From the Process to Prepare 1,1,3,3-tetrachloropropene, 1,3,3,3-tetrachloropropene, or Combinations Thereof

The 1,1,1,3,3-pentachloropropane fed to the above described process may be converted to 1,1,3,3-tetrachloropropene, 1,3,3,3-tetrachloropropene, or combinations thereof in at least 50% conversion. In various embodiments, the conversion of 1,1,1,3,3-pentachloropropane to 1,1,3,3-tetrachloropropene, 1,3,3,3-tetrachloropropene, or combinations thereof may be at least 50%, at least 60%, at least 75%, at least 85%, at least 95%, and at least 99%.

The selectivity to 1,1,3,3-tetrachloropropene, 1,3,3,3-tetrachloropropene, or combinations thereof can and will vary depending on the reaction conditions, base, the purity level of the 1,1,1,3,3-pentachloropropane, and the 1,1,3,3-tetrachloropropene, 1,3,3,3-tetrachloropropene, or combinations thereof produced. Generally, the selectivity to 1,1,3,3-tetrachloropropene, 1,3,3,3-tetrachloropropene, or combinations thereof may be greater than 70%. In various embodiments, the selectivity to the 1,1,3,3-tetrachloropropene, 1,3,3,3-tetrachloropropene, or combinations thereof may be greater than 70%, greater than 80%, greater than 90%, or greater than 95%. In preferred embodiments, the selectivity to the 1,1,3,3-tetrachloropropene, 1,3,3,3-tetrachloropropene, or combinations thereof may range from 95% to 99%.

(d) Separation of the 1,1,3,3-tetrachloropropene, 1,3,3,3-tetrachloropropene, or Combinations Thereof and Recycling Product Streams

The process for separating the 1,1,3,3-tetrachloropropene, 1,3,3,3-tetrachloropropene, or combinations thereof from the reactor contents is described above in Section (II). Specific recycle streams useful in improving the efficiency of the process are described above in Section (II).

The product effluent stream from the separator comprising the 1,1,3,3-tetrachloropropene, 1,3,3,3-tetrachloropropene, or combinations thereof produced in the dehydrochlorination process may have a yield of at least about 10%. In various embodiments, the product effluent stream comprising 1,1,3,3-tetrachloropropene, 1,3,3,3-tetrachloropropene, or combinations thereof produced in the process may have a yield of at least about 20%, at least about 50%, at least about 70%, at least about 75%, at least about 80%, at least about 85%, at least about 90%, at least about 95%, or at least about 99%.

Patent

### (VI) Preferred Embodiment: Process for Preparing of 1,1,2,3-Tetrachloropropene

(a) process for Preparing 1,1,2,3-tetrachloropropene

In another aspect, disclosed herein are processes for preparing 1,1,2,3-tetrachloropropene. The process commences by preparing and reacting a mixture comprising 1,1,2,2,3-pentachloropropane; an aqueous base; and a phase transfer catalyst. The phase transfer catalyst is described above in Section (I)(b) and the aqueous base is described above in Section (I)(c). In a preferred embodiment, the phase transfer catalyst is trioctylmethylammonium chloride (Aliquat 336) and the aqueous base comprises 5 to 10 wt % NaOH, KOH, or combinations thereof and up to or below the saturation limit of the corresponding salt.

(b) Reaction Conditions

The reaction conditions are described above in Section (I)(d).

(c) Output From the Process to Prepare 1,1,2,3-tetrachloropropene

The 1,1,2,2,3-pentachloropropane fed to the above described process may be converted to 1,1,2,3-tetrachloropropene in at least 50% conversion. In various embodiments, the conversion of 1,1,2,2,3-pentachloropropane to 1,1,2,3-tetrachloropropene may be at least 50%, at least 60%, at least 75%, at least 85%, at least 95%, or at least 99%.

The selectivity to 1,1,2,3-tetrachloropropene can and will vary depending on the reaction conditions, base, the purity level of the 1,1,2,2,3-pentachloropropane used as a starting material. Generally, the selectivity to 1,1,2,3-tetrachloropropene, may be greater than 70%. In various embodiments, the selectivity to the 1,1,2,3-tetrachloropropene, may be greater than 70%, greater than 80%, greater than 90%, or greater than 95%. In preferred embodiments, the selectivity to the 1,1,2,3-tetrachloropropene may range from 95% to 99%.

(d) Separation of the 1,1,2,3-tetrachloropropene, and Recycling Product Streams

The process for separating the 1,1,2,3-tetrachloropropene from the reactor contents is described above in Section (II). Specific recycle streams useful in improving the efficiency of the process are described above in Section (II).

The product effluent stream from the separator comprising the 1,1,2,3-tetrachloropropene produced in the dehydrochlorination process may have a yield of at least about 10%. In various embodiments, the product effluent stream comprising 1,1,2,3-tetrachloropropene produced in the process may have a yield of at least about 20%, at least about 50%, at least about 70%, at least about 75%, at least about 80%, at least about 85%, at least about 90%, at least about 95%, or at least about 99%.

### (VII) Further Reaction of the Chlorinated Alkenes

In one aspect, disclosed herein are processes for the conversion of halogenated alkenes, such as 1,1,3-trichloropropene, 3,3,3-trichloropropene, or combinations thereof to one or more highly chlorinated alkanes, such as 1,1,1,2,3-pentachloropropane (240DB) by reaction with a chlorinating agent, such as chlorine.

In another aspect, disclosed herein are processes for the conversion of halogenated alkenes, such as 1,1,2,3-tetrachloropropene, 2,3,3,3-tetrachloropropene, or combinations thereof to one or more highly chlorinated alkanes by reaction with a chlorinating agent, such as chlorine.

In yet another aspect, disclosed herein are processes for the conversion of halogenated alkenes, such as 1,1,3,3-tetrachloropropene, 1,3,3,3-tetrachloropropene, or combinations thereof, to one or more highly chlorinated alkanes by reaction with a chlorinating agent, such as chlorine.

In yet another aspect, disclosed herein are processes for the conversion of halogenated alkenes, such as 1,1,2,3-tetrachloropropeneto one or more highly chlorinated alkanes by reaction with a chlorinating agent, such as chlorine.

In another aspect, disclosed herein are processes for the conversion of halogenated alkenes, such as 1,1,3-trichloropropene, 3,3,3-trichloropropene, or combinations thereof; 1,1,2,3-tetrachloropropene, 2,3,3,3-tetrachloropropene, or combinations thereof; and 1,1,3,3-tetrachloropropene, 1,3,3,3-tetrachloropropene, or combinations thereof; and 1,1,2.3-tetrachloropropene to one or more of the above described chlorinated alkanes, which may then be converted to hydrofluoroolefins.

These processes comprise contacting the halogenated alkanes and/or halogenated alkenes with a fluorinating agent in the presence of a fluorination catalyst, in a single reaction or two or more reactions. These processes can be conducted in either gas phase or liquid phase with the gas phase being preferred at temperatures ranging from 50° C. to 400° C.

Generally, a wide variety of fluorinating agents can be used. Non-limiting examples of fluorinating agents include HF, F₂, CIF, AlF₃, KF, NaF, SbF₃, SbF₅, SF₄, or combinations thereof. The skilled artisan can readily determine the appropriate fluorination agent and catalyst. Examples of hydrofluoroolefins that may be produced utilizing these processes include, but are not limited to 2,3,3,3-tetrafluoroprop-1-ene (HFO-1234yf), 1,3,3,3-tetrafluoroprop-1-ene (HFO-1234ze), 3,3,3-trifluoroprop-1-ene (HFO-1243zf), and 1-chloro-3,3,3-trifluoroprop-1-ene (HFCO-1233zd).

FIG. 2 illustrates one possible configuration of components that can be used to perform the processes disclosed herein. The starting materials, as described above, are combined in the reactor, where the reaction occurs. While FIG. 2 shows the use of A336 (Aliquat 336), 250FB, NaOH and H₂O, other reagents and materials, as disclosed herein, may be used. And while FIG. 2 shows the A336 being added to the 250FB, NaOH, and H₂O stream, this is not the only way of adding the A336, i.e., the A336 may be added directly to the reactor. The reaction product then leaves the reactor and enters a separator, where at least a portion of the aqueous layer, which contains NaCl, is removed. The organic layer then leaves the decanter and enters a separation column, T1, where heavies are separated and leave T1. All or some of the heavies may be discarded, recycled or both recycled and discarded. The organic material leaving T1 may enter a second separation column, T2. In T2 the desired alkenes, such as 113e and 333e are isolated, while any heavies are optionally recycled to the reactor or discarded (not shown). T1 and T2 may be any type of distillation column that is known in the art, and they may be the same or different. If desired, additional distillations or purifications may be performed to obtain material of a desired purity.

### DEFINITIONS

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “1113e” refers to 1,1,3-trichloropropene.

The term “333e” refers to 3,3,3-trichloropropene.

The term “1123e” refers to 1,1,2,3-tetrachloropropene.

The term “2333e” refers to 2,3,3,3-tetrachloropropene.

Having described the invention in detail, it will be apparent that modifications and variations are possible without departing from the scope of the invention defined in the appended claims.

### EXAMPLES

The following examples illustrate various embodiments of the invention.

### Example 1: Preparation of 1,1,3-Trichloropropene and 3,3,3-Trichloropropane Using Fresh Aliquat 336 at 700 rpm (Run 1).

Into a round bottom flask was introduced 250FB (1,1,1,3-tetrachloropropane, 87 g), fresh Aliquat 336 (1.57 g), and an aqueous solution of 8 wt % caustic solution containing 16 wt % of NaCl (225 g). Stirring of the mixture commenced at 700 rpm, then the mixture was heated at 60° C. under atmospheric pressure. Aliquots of the mixture were removed and analyzed by GC at specific time intervals. From this data, plots of % selectivity versus % conversion (FIG. 1) and % conversion versus time (FIG. 3) were generated. In FIGS. 1 and 3, the data from this example is denoted as Run 1. In FIGS. 1 and 3, Runs 22, 23, and 26 use recycled phase transfer catalyst (Aliquat 336), whereas Runs 25 and 1 use fresh Aliquat 336.

### Example 2: Preparation of 1,1,3-Trichloropropene and 3,3,3-Trichloropropane Using Fresh Aliquat 336 at 900 rpm (Run 25)

Into a round bottom flask was introduced 250FB (1,1,1,3-tetrachloropropane, 87 g), fresh Aliquat 336 (1.57 g), and an aqueous solution of 8 wt % caustic solution containing 16 wt % of NaCl (225 g). Stirring of the mixture commenced at 900 rpm, then the mixture was heated at 60° C. under atmospheric pressure. Aliquots of the mixture were removed and analyzed by GC at specific time intervals. From this data, plots of % selectivity versus % conversion (FIG. 1) and % conversion versus time (FIG. 3) were generated. In FIGS. 1 and 3, the data from this example is denoted as Run 25.

### Example 3: Preparation of 1,1,3-Trichloropropene and 3,3,3-Trichloropropane Using Recycled Aliquat 336 at 700 rpm (Run 22)

Into a round bottom flask was introduced 250FB (1,1,1,3-tetrachloropropane, 87 g), recovered Aliquat 336 with heavy by-products after removal of 113e and 333e by distillation at reboiler temperature of 160-180° C. (1.7 g), and an aqueous solution of 8 wt% caustic solution containing 16 wt % of NaCl (225 g). Stirring of the mixture commenced at 700 rpm, then the mixture was heated at 60° C. under atmospheric pressure. Aliquots of the mixture were removed and analyzed by GC at specific time intervals. From this data, plots of % selectivity versus % conversion (FIG. 1) and % conversion versus time (FIG. 3) were generated. In FIGS. 1 and 3, the data from this example is denoted as Run 22.

### Example 4: Preparation of 1,1,3-Trichloropropene and 3,3,3-Trichloropropane Using Recycled Aliquat 336 at 700 rpm (Run 23)

Into a round bottom flask was introduced 250FB (1,1,1,3-tetrachloropropane, 87 g), recovered Aliquat 336 with heavy by-products after removal of 113e and 333e by distillation at reboiler temperature of 160-180° C. (2.0 g), and an aqueous solution of 8 wt % caustic solution containing 16 wt % of NaCl (225 g). Stirring of the mixture commenced at 700 rpm, then the mixture was heated at 60° C. under atmospheric pressure. Aliquots of the mixture were removed and analyzed by GC at specific time intervals. From this data, plots of % selectivity versus % conversion (FIG. 1) and % conversion versus time (FIG. 3) were generated. In FIGS. 1 and 3, the data from this example is denoted as Run 23.

### Example 5: Preparation of 1,1,3-Trichloropropene and 3,3,3-Trichloropropane Using Recycled Aliquat 336 at 900 rpm (Run 26)

Into a round bottom flask was introduced 250FB (1,1,1,3-tetrachloropropane, 87 g), recovered Aliquat 336 with heavy by-products after removal of 113e and 333e by distillation at reboiler temperature of 160-180° C. (2.0 g), and an aqueous solution of 8 wt % caustic solution containing 16 wt % of NaCl (225 g). Stirring of the mixture commenced at 900 rpm, then the mixture was heated at 60° C. under atmospheric pressure. Aliquots of the mixture were removed and analyzed by GC at specific time intervals. From this data, plots of % selectivity versus % conversion (FIG. 1) and % conversion versus time (FIG. 3) were generated. In FIGS. 1 and 3, the data from this example is denoted as Run 26.

### Data Evaluation From the Above Examples

The data from each individual experiment was plotted separately in FIGS. 1 and 3. As FIG. 1 shows, the data indicates recycled Aliquat 336 with heavy by-products showed similar selectivity as fresh Aliquat 336. Therefore, recycling the heavy by-products can be used to significantly reduce the use of fresh Aliquat 336 and thus provides increased efficiency and cost savings in the process.

FIG. 3 shows the reaction rate utilizing recycled heavy by-products containing Aliquat 336 or fresh Aliquat 336 phase transfer catalyst. As FIG. 3 shows, the reaction rate using recycled heavy by-products containing Aliquat 336 was faster than using fresh Aliquat 336 and thus results in an increase in reactor productivity or a reduction in capital cost.

What is claimed is:

1. A process for producing at least one chlorinated alkene, the process comprising:
a) preparing and reacting a mixture comprising at least one chlorinated alkane, an aqueous base, and a phase transfer catalyst, wherein the mixture comprises an aqueous phase and an organic phase comprising the catalyst;
b) separating at least some of the organic phase from the aqueous phase, and optionally drying the organic phase;
c) distilling at least part of the organic phase in at least one distillation column to produce two product effluent streams wherein product effluent stream
(i) comprises the at least one chlorinated alkene and optionally unreacted chlorinated alkane and the product effluent stream (ii) comprises the heavy by-products, the phase transfer catalyst, optionally, unreacted chlorinated alkane; and
d) recycling at least a portion of the product effluent stream (ii) to step a).

2. The process of claim 1, wherein product effluent stream (i) from step (c) is further distilled to produce two additional product effluent stream, an overhead stream comprising the chlorinated alkene and a bottom stream comprising unreacted chlorinated alkane; and recycling a portion of the bottom stream to step a).

3. The process of claim 1, wherein the product effluent stream (ii) from step c) is further distilled to produce two additional product effluent streams, an overhead stream comprising the unreacted chlorinated alkane and a bottom stream comprising the heavy by-products and the phase transfer catalyst; and recycling at least a portion of the overhead stream and a portion of the bottom stream to step a).

4. The process of claim 1, wherein less than 98% of the chlorinated alkane is converted into the chlorinated alkene.

5. The process of claim 1 wherein the chlorinated alkane is a chlorinated propane, and the chlorinated propane comprises a dichlorinated propane, a trichlorinated propane, a tetrachlorinated propane, a pentachlorinated propane, a hexachlorinated propane, or combinations thereof.

6. The process of claim 1, wherein the chlorinated propane comprises at least one of 1,1,1,3-tetrachloropropane (250FB); 1,1,1,2,3-pentachloropropane (240DB) or 1,1,1,3,3-pentachloropropane (240FA).

7. The process of claim 1, wherein the chlorinated propene comprise mono chlorinated propene, a dichlorinated propene, a trichlorinated propene, a tetrachlorinated propene, a pentachlorinated propene, or combinations thereof.

8. The process of claim 7, wherein the chlorinated propene comprises 1,1,3-trichloropropene; 3,3,3-trichloropropene, or combinations thereof; or the chlorinated propene comprises 1,1,2,3-tetrachloropropene, 2,3,3,3-tetrachloropropene, or combinations thereof; or the chlorinated propene comprises 1,1,3,3-tetrachloropropene, 1,3,3,3-tetrachloropropene, or combinations thereof.

9. The process of claim 1, wherein the aqueous base comprises an aqueous solution of sodium hydroxide, potassium hydroxide, lithium hydroxide, calcium hydroxide, sodium carbonate, potassium carbonate, sodium bicarbonate, potassium bicarbonate, or combinations thereof.

10. The process of claim 1, wherein the aqueous base comprises an aqueous solution of sodium hydroxide.

11. The process of claim 1, wherein the phase transfer catalyst comprises a quaternary ammonium salt, a quaternary phosphonium salts, or combinations thereof.

12. The process of claim 1, wherein the phase transfer catalyst is methyltrioctylammonium chloride (Aliquat 336).

13. The process of claim 1, wherein the concentration of the phase transfer catalyst in the chlorinated alkane is from 0.001 wt % to about 10.0 wt %.

14. The process of claim 1, wherein the phase transfer catalyst comprises a fresh liquid feed, at least a portion of the product effluent stream, or combinations thereof.

15. The process of claim 1, wherein the selectivity of the process is at least 70%.

16. The process of claim 1, wherein the conversion of the process is at least 50%.

17. The process of claim 1, wherein the mixture is stirred by jet mixing, and the jet mixing is caused by feeding fresh liquid feed, product effluent stream, a recycle stream or combinations thereof to at least one nozzle;
wherein the fresh liquid feed comprises the chlorinated alkane, the phase transfer catalyst, and/or an aqueous base;
wherein the product effluent stream comprises phase transfer catalyst;
wherein a portion of the product effluent stream is recycled to the reactor in a recycle stream.

18. The process of claim 1, wherein the recycle stream or a portion of the recycle stream is heated using a heat exchanger to maintain the desired reaction temperature.

19. The process of claim 1, wherein the fresh liquid feed comprising non-recycled chlorinated alkane, phase transfer catalyst, and/or aqueous base, has a fresh liquid feed mass flow, the recycle stream of step d) has a recycle stream mass flow, and the ratio of the recycle stream mass flow to the fresh liquid feed mass flow is adjusted to maintain the reaction conversion.

20. The process of claim 1, wherein the aqueous base further comprises NaCl, KCl, CaCl₂, or combinations of two or more thereof.
